(19) United States
(12) Patent Application Publication
(10) Pub. No.: US 2007/0219587 A1
(43) Pub. Date: Sep. 20, 2007

(54) Surgical Thread for Plastic, Dermatologic, and Reconstructive Surgery and for Surgical Sutures

(75) Inventor: Ciro Accardo, Portici (NA) (IT)

Correspondence Address: YOUNG & THOMPSON, 745 SOUTH 23RD STREET, 2ND FLOOR, ARLINGTON, VA, 22202, US

(73) Assignee: Promoitalia International SRL, Napoli (IT)

(21) Appl. No.: 10/594,087

(22) PCT Filed: Dec. 6, 2005
(86) PCT No.: PCT/IT05/00716

§ 371 Date: Sep. 25, 2006

(30) Foreign Application Priority Data
Dec. 6, 2004 (IT) RM2004A000599

(51) Int. Cl. A61L 17/00

(52) U.S. Cl. 606/228

(57) ABSTRACT

A surgical thread to be used for plastic, dermatological, cosmetic, operations as well as for general surgery, emergency surgery, and all of specialized surgeries is provided with inclined projections or thorns or hooks or protrusions that have manifold shapes and predetermined size and are distributed along a spiral as well as are arranged in sequences which are alternately inclined to opposed directions so as to prevent the thread from slipping or moving to both opposed directions.

The present invention relates to medicine and means for general surgical operations such as plastic, cosmetic, dermatological surgery. In particular, this invention relates to a surgical thread to be used for plastic, cosmetic, dermatological operations as well as for general surgery, emergency surgery, and all of specialized surgeries. The innovative feature of the thread of this invention consists of inclined projections (thorns) or hooks or protrusions having various shape and predetermined size and varying within a defined range as far as their technical parameters is concerned, and spatially distributed along a spiral unlike the state of art.

Such a result is accomplished by a straight thread provided with thorns distributed according to a determined sequence along the axis of the thread and forming one or more spirals that run to the same or opposite directions.

A second feature of the finding is that the section of the thread is not constant apart its shape and has an increasing or decreasing diameter.

A third feature of the finding is that the thread is not straight but induced to take spatially a permanent spiral shape with a well defined inner diameter, this giving it a peculiar elastic behaviour (like a spring). Thus, the opposite thorns can also be distributed along the same axis.

A fourth feature of the finding is that the pitch of the thorn along the spiral thread, i.e. the distance between two thorns, can vary with well defined parameters.

A fifth feature of the finding is that the thorns are not smooth but take the shape of a fishbone or an ear of wheat and/or develop like a microspiral.

A sixth feature of the finding is that the thread provided with thorns is wrapped up by a spiral thread with a lower diameter, the inner gauge of the spiral thread being at least equal to the diameter of the needle by which the disclosed thread is inserted into the tissues.

Such features characterize a fully new parameter of the surgical threads, i.e. the repetition unit, which is the portion of thread between two left and right ends with a determined length (that can vary from 2 cm to 60 cm) with a middle point Pm at which the thorns invert their own inclination giving rise to two opposite sequences of thorns. In other words, all of the thorns of one sequence are directed from left to middle point Pm in the segment running from left to middle point Pm, while all of the thorns of the other sequence are directed from right to middle point Pm in the segment running from right to middle point Pm, or both sequences of thorns are directed from middle point Pm towards the respective ends.

A seventh feature of the finding is the provision of repetition units with different lengths in a thread of determined length so as to provide a differentiated gathering according to the type of soft tissue crossed. For example, repetition units with different lengths, i.e. 5 cm, 12 cm, 22 cm, etc., can alternate over a thread 1 metre long according to a sequential, symmetric or asymmetric, order relative to the total length of the thread.

A further advantage of the finding is that the above-mentioned features can be combined with one another such as to provide a surgical thread which is responsive to the particular requirements of the operator and a correct medical, surgical mode of use. Thus, for example, there is provided a linear thread carrying two or three spirals on its outside surface having design parameters such as pitch of the turns, number of turns per unit length, etc., such thread being also able to be wound like a spiral about a cylinder with a specific diameter and to be treated by known chemical-physical methods to induce a permanent shape memory giving it peculiar elastic properties. All of the technical features as well as the different shapes mentioned above can be accomplished by using a die-casting production technique (using both permanent and reabsorbable materials with very long duration) or by applying nanotechnology techniques. The shapes of the projections or indentations such as thorns, fishbone, ears of wheat, spirals or other geometrical figures can thus be selected among a very high number of combinations designed for the desired specific function.

A prior European Patent Application n. EP 1 075 843 discloses a surgical thread for plastic surgery operations having inclined projections extending from one end all over the length of the thread except for a short length at the opposite end where the projections are inclined to the opposed direction. Such projections have the peculiarity of being produced on the shape of conical hooks so as to prevent the thread from slipping or moving in the presence of a tensile stress exerted on both thread ends. Reference should be made to those forces due to the dynamic actions of voluntary or involuntary muscles such as those of facial expressions, mastication, deambulation, limb movement, trunk torsion, etc.

Therefore, the presence of such formations on the thread axis prevents the latter from being moved both to the right and left (if the ends of the thread were drawn) once it is inserted into the soft tissues of the human body and its ends appear from the skin surface or are immersed into the hypodermic thickness of the human skin or to the level of the connective fasciae or muscles.

A recent application WO 03/103972 discloses a surgical thread of metallic, polymeric or biological material where the projections or thorns are distributed all over the length of the thread on one or more sides of the front section of the latter with alternate sequences of inclination.

If this solution allows on one hand the lifting and the durable stretching and fastening of the soft tissues to a new position to be handled, on the other hand it still has some drawbacks. First of all attention should be paid in the production of the projections as they have to withstand the maximum tensile stress of the thread, and an increase in the number of thorns per unit length according to such a solution is greatly to the detriment of the strength of the thread.

Furthermore, if the thread is used for the lifting of the soft tissues, the most important things for the lifting to produce correct effects is the right length of the thorns and a still more exact angle included with the longitudinal axis, without causing a reduction in the tensile strength of the thread.

The present invention seeks to overcome the above-mentioned problems by providing a surgical thread wherein a suitable distribution of the thorns or better the stereotaxic orientation of the thorns in the three-dimensional space and relative to the axis of the thread as well as the selection of some significant, structural, functional parameters of the thorns guarantee a tissular fibrosis suitable for a steady repositioning and lifting of the soft tissues which is durable in time, while keeping the tensile strength of the thread unchanged.

A better understanding of the invention will result by referring to the accompanying drawings that show some preferred embodiments thereof only by way of a not limiting example.

In the drawings:

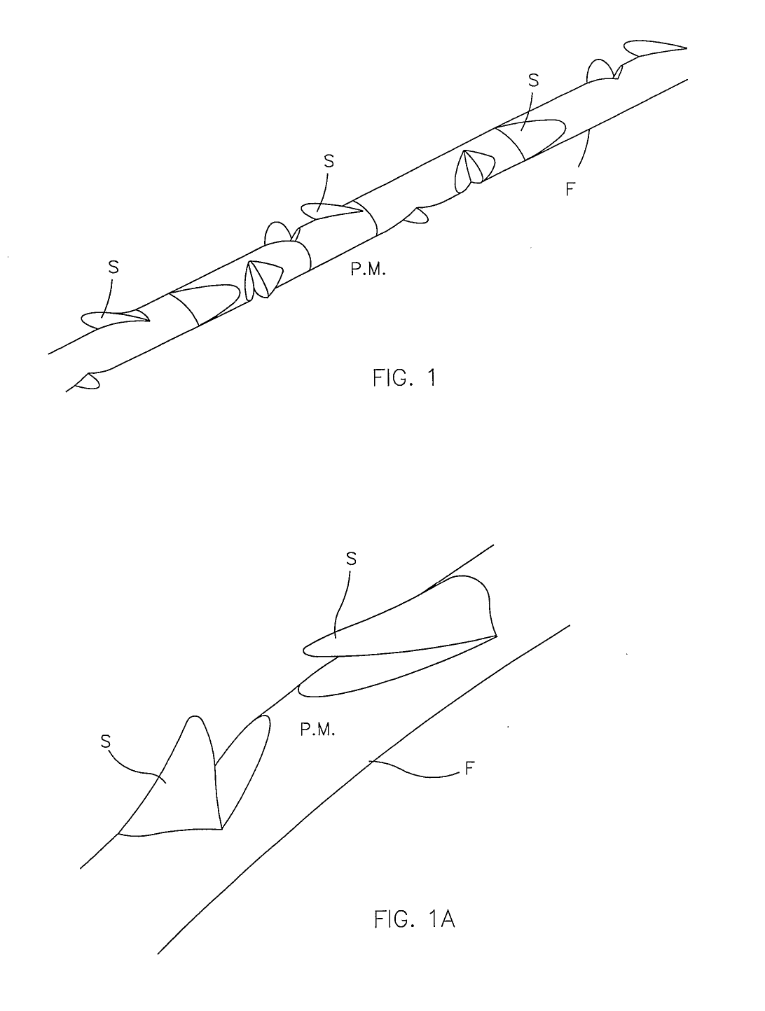

FIG. 1 shows a surgical thread according to the invention which is straight and provided with projections or opposed thorns distributed according to a spiral;

FIG. 1A is a detail of FIG. 1 where the middle point of the repetition unit as described thereafter is shown;

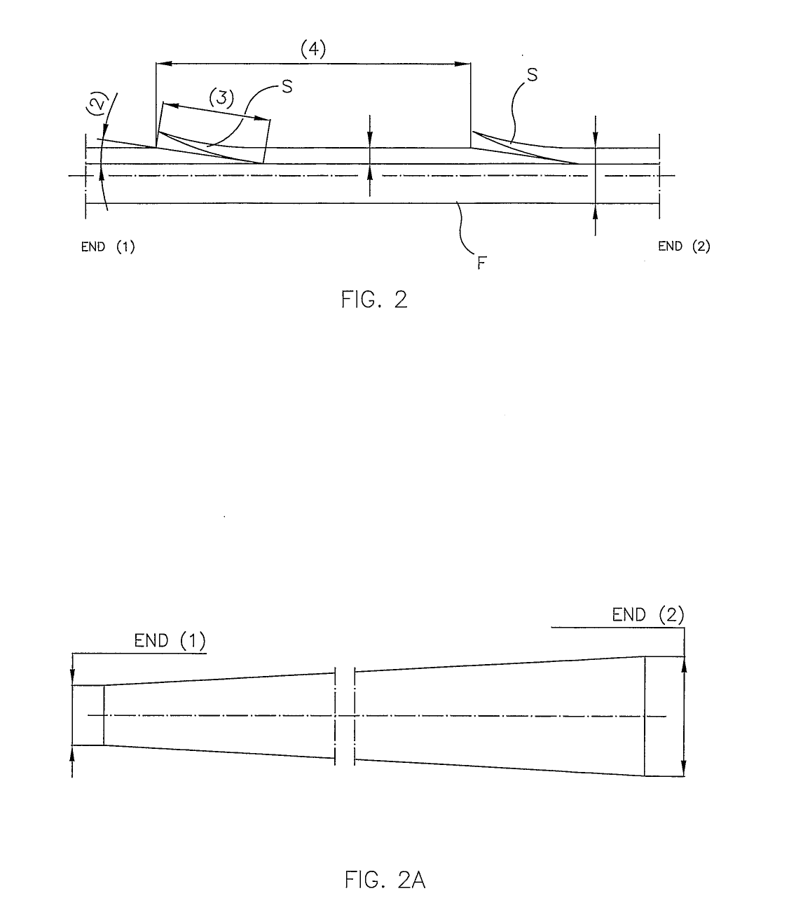

FIG. 2 is a section view of a length of the straight thread which shows up the formation of the thorns;

FIG. 2A shows a section of a length of the thread with a diameter increasing towards one end;

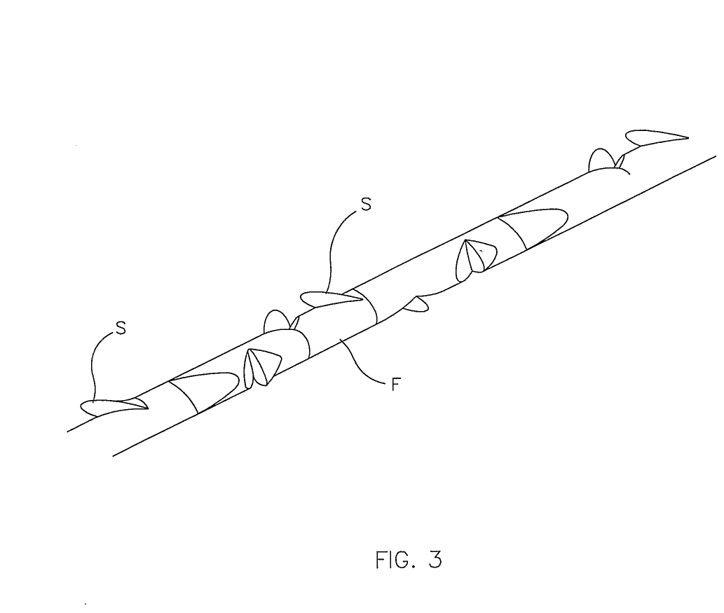

FIG. 3 shows a straight thread with projections distributed according to two spirals and directed to opposed directions, one from the left to the right in left-handed direction, the other from the right to the left in right-handed direction;

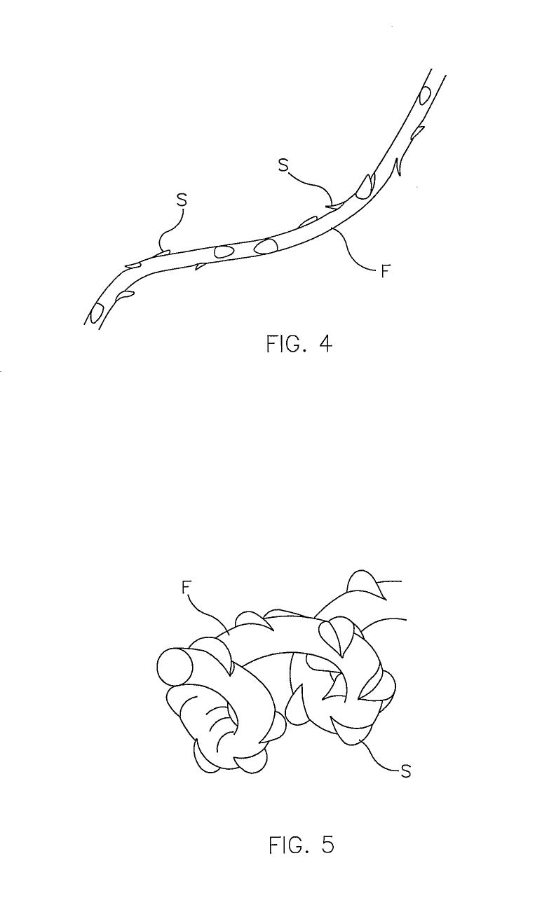

FIG. 4 shows a non-straight thread provided with thorns distributed along spirals extending to opposed directions, wherein the spiral shape of the thread ensures an increasing number of thorns per unit length of the thread as a function of the diameter and the distance of the turns;

FIG. 5 is a three-dimensional view of a spiral thread provided with two spirals of thorns shifted 180° out of phase to each other;

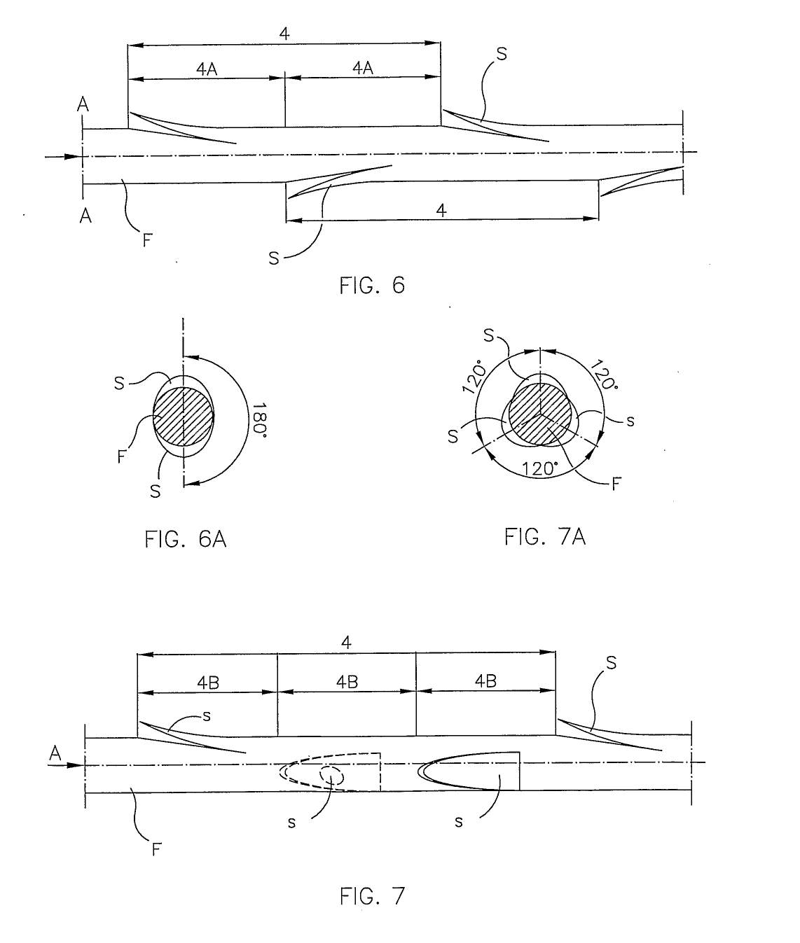

FIG. 6 shows in detail a section of a thread portion provided with thorns positioned along two spirals shifted 180° out of phase to each other;

FIG. 6A is a section view according to the plane A-A of FIG. 6;

FIG. 7 shows in detail a sectioned portion of the straight thread having thorns positioned on two spirals shifted 120° out of phase to each other;

FIG. 7A is a section view according to the plane A-A of FIG. 7;

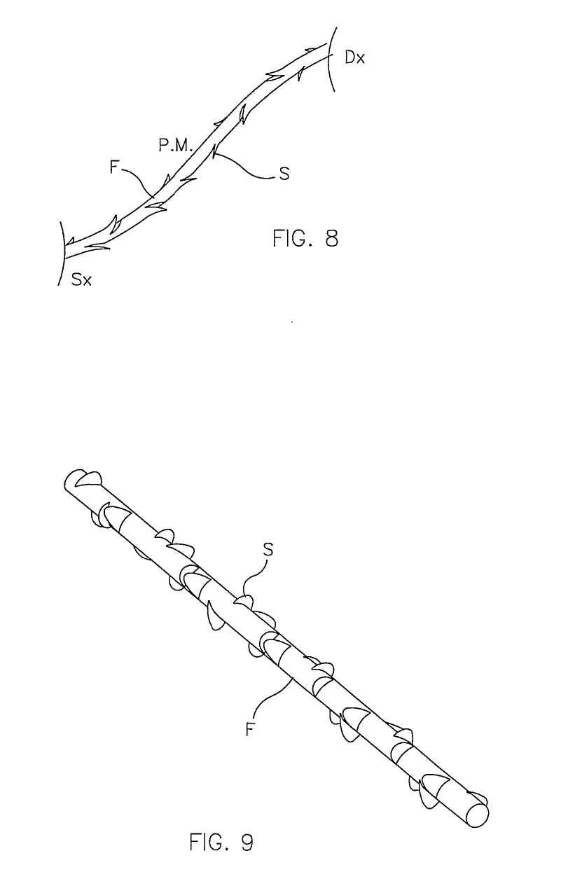

FIG. 8 is a three-dimensional view of a straight thread provided with thorns positioned on three spirals shifted 120° out of phase to one another and middle point;

FIG. 9 shows a straight thread with four spirals provided with thorns, two of them being right-handed and the other two left-handed;

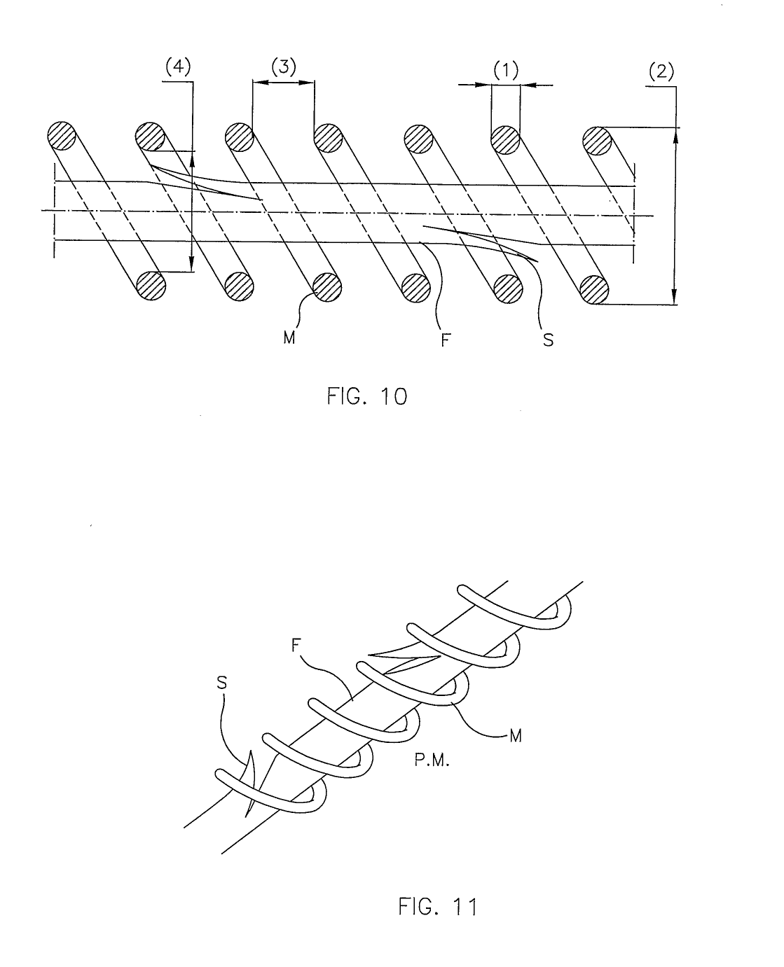

FIG. 10 shows in detail a longitudinal section of a spiral thread portion;

FIG. 11 shows a perspective view of a spiral thread portion that shows up the opposed thorns at the middle point of the inner thread;

FIG. 12 shows an embodiment of a thread with four spirals of thorns.

With reference to FIG. 1, a surgical thread F for plastic surgery operations has a plurality of projections or thorns S inclined to opposed directions and placed in sequence all over the length of the thread, except for short segments connecting a repetition unit, indicated at U, to the next unit.

As previously indicated, a repetition unit is defined as the portion of the thread included between two left and right ends with a determined length varying from 2 cm to 60 cm and having a middle point with respect to which the thorns are directed to opposed directions, i.e. they are directed from the left to the middle point in the segment running from the left to the middle point, and from the right to the middle point in the segment running from the right to the middle point, or both thorns are directed to the opposed direction (see FIG. 8). With recurring unit a whole number of repetition units on the thread is meant.

The repetition units can have the same pitch or length or different pitches or lengths varying from a minimum length of 2 cm to a maximum length of 60 cm.

The number of recurring units depends on the total length of the thread on which they are placed and the length of the repetition units that will recur regularly all over the length of the thread. For example, a 1 metre long thread having 5 cm long repetition units will carry 20 recurring units.

Protrusions or thorns S shown in detail in FIG. 2 have the shape of truncated cone but can also take different shapes, for example conical, half-conical, fishbone, ear of wheat, microspiral, etc. Such projections can be designed either with sharp endings or without cusps, either flexible, elastic or rigid.

Such protrusions are provided with a well-defined cutting angle indicated at 2 in FIG. 2 which corresponds to the angle that the tangent to the thorn in its connection point to the thread includes with the parallel to the axis of the thread crossing the same point, such angle being between 10° and 70°.

The length 3 of such thorns or projections is varying between 0.3 mm and 5.00 mm but can also be greater. Pitch 4, i.e. the distance of the connecting points among thorns and thread, can be constant between 0.50 mm and 17.20 mm or varying according to the Fibonacci's series referred to the order of magnitude of the thread's thickness. While in FIG. 1 the diameter of the thread is constant preferably between 0.10 mm and 4 mm, FIG. 2A shows a thread with an increasing diameter between end 1 and end 2. In this case the minimum diameter will preferably be between 0.05 mm and 0.50 mm, while the maximum diameter will be between 3.00 and 7.00 mm.

In a first embodiment shown in FIG. 1, thorns S are placed like a spiral along the greater axis of the thread.

In a second embodiment shown in FIG. 3, divergent or opposed thorns S are distributed along two opposed spirals having opposed directions, one right-handed spiral directed to the clockwise direction from the right end to the left end for an observer looking frontally the right end of the thread, and the other left-handed spiral directed to the anti-clockwise direction from the left end to the right end for an observer looking frontally the left end of the thread. It should be noted that a thread of this type having a greater number of projections or thorns in each repetition unit features a number of thorns per unit length (i.e. interaction areas with human soft tissues) that can be two times as much in threads provided with two opposed spirals shifted 180° out of phase, as seen in FIG. 6 (where the pitch of the thorns on both spirals is indicated at 4A) or be three times as much in threads provided with three spirals shifted 120° out of phase to one another (see FIG. 7 where the pitch of the thorns on the three spirals is indicated at 4B), or be four times as much in threads provided with four spirals shifted 90° out of phase. The density of the thorns can still be greater if they develop on a thread with increasing diameter having single, double or triple or quadruple spirals ensuring gathering, lifting and holding actions of the soft tissues or the surgical suture edges which are far greater than any other pre-existent similar structure. Moreover a further doubling of the gathering can be obtained if the thorns of the repetition unit are placed on two or more opposed, left-handed and right-handed spirals developing from the right and left ends of the thread and terminating not at the middle point but at the other left or right end of the repetition unit or the thread with determined length, respectively.

In a different embodiment the spatial development of the thread is not straight but can have a spiral course with a precise inner gauge between 3 mm and 1.72 cm and a well-defined distance among turns between 0 mm and 1.30 cm and a spiral arrangement (see FIG. 4) according to the formula of the logarithmic Fibonacci's series spiral with an additional constant X, or according to the formula of the logarithmic spiral with constant radius and pitch, or according to the formula of the logarithmic spiral with constant radius and pitch of the modified Fibonacci's series, or according to other numerical series.

The spiral thread can be modified or not at its outside surface according to the above-mentioned specification and have thorns with straight or spiral course.

Preferably, the spiral shape is obtained by inserting first the thread having the technical features disclosed above into the core of a needle (gauge varying from 12 gauge to 21 gauge) and next winding the portion appearing from its end like a spout of flute about the needle. Then the spiral thread is treated by chemical-physical methods to induce a permanent shape memory so that it can act like a spring.

Alternatively, such thread provided with thorns distributed according to one or more spirals can be wound about a virtual cylinder with a varying specific diameter and then treated as disclosed above to provide a permanent shape giving it peculiar elastic characteristics, i.e. a further increase in the density of the thorns per unit length.

Advantageously, threads with this shape can also be inserted in those soft tissues or other living tissues of different species (for example for the suture or suspension of organs in veterinary science) that are subject to tissue movement or strong dynamic actions as the spiral thread can either take more compact or extended shapes for time periods defined by the dynamic action, thus acting as the spring of a car damper.

According to a further embodiment shown in FIGS. 10 and 11, a spiral thread M with lower diameter is wound about thread F provided with thorns S, the inner gauge of the spiral being at least the same as the diameter of the needle by which the disclosed thread is inserted into the tissue.

It should be noted that the thread of the invention can be made of metallic, or polymeric, or biological or synthetic material. In particular, it can be made of reabsorbable material with a very long duration, with the undeniable advantages involved.

The method of use of the thread is as follows.

The thread is inserted into the hypoderm by means o a guide needle (gauge 0.9-1.5 mm, length 12-18 mm).

The operation technique consists of giving in advance a topic anaesthesia with 1-2-3% xylocalne or carbocaine with or without the addition of 1/200000 diluted adrenaline by an infiltration of not more than 0.5-0.7 ml per unit administering. The guide needle is caused to slide into the hypoderm following the course marked in advance on the skin and to appear from the predetermined point. The thread of the invention with its convergent Fibonacci's spiral thorns is inserted into the core of the guide needle.

Then, after the plicate skin is seized between forefinger and thumb of the left hand of a right-handed surgeon (thus determining the desired hypercorrection in the area of the face or the body to be treated), the needle is removed and the thread remains in situ giving the desired plasticity to the tissues. Afterwards, the ends of the thread are first pulled so that each thorn comes to an engagement and then the ends of the thread are cut and caused to sink into the hypoderm.

The indications for the operation by this new method are as follows: ptosis of the face, ptosis of the neck, tissular softening, flattened face and not very protruding face contours, correction of the iatrogenic, congenital or traumatic paresis of the facial nerve, bosom hypotonicity, hypotonicity of the axillary region, hypotonicity and dermatoclasy of the gluteal region, inner thigh, crural groin, pelvic pavement, and other regions of the body, etc.

In conclusion, the use of a surgical thread to support and lift the soft tissues of the face and the neck allows the operation time to be reduced and does not leave or produce any scar visible on the skin.

Additionally, it guarantees a quite natural face for a time from 5 to 7 years but most of all above suspicion of a traditional lifting which is always visible.

At last, it should be appreciated that such modified threads have a regenerative trophic effect to the human tissues in which they are inserted, as proved by a number of documents of scientific studies. In conclusion, such thread can also be used for the suspension, repositioning, connective regeneration and rejuvenation of human soft tissues.

The present invention has been described according to some preferred embodiments thereof. However, it is self-evident that those skilled in the art can make a number of modifications and/or variations without departing from the scope of the present invention as defined in the appended claims.

1. A surgical thread to be used for plastic, dermatological, cosmetic, operations as well as for general surgery, emergency surgery, and all of specialized surgeries, characterized in that it is provided with inclined projections or thorns or hooks or protrusions that are distributed along at least one spiral.

2. The surgical thread according to claim 1, characterized in that said projections or thorns are arranged in sequences which are alternately inclined to opposed directions so as to prevent the thread from slipping or moving to both opposed directions.

3. The surgical thread according to claim 1, characterized in that said divergent or opposed thorns or protrusions are distributed along two opposed spirals having opposed directions, one right-handed spiral directed to the clockwise direction from the right end to the left end, and the other left-handed spiral directed to the anti-clockwise direction from the left end to the right end.

4. The surgical thread according to claim 1, characterized in that the thorns are distributed over three spirals shifted 120° out of phase to one another.

5. The surgical thread according to claim 1, characterized in that the thorns are distributed according to four spirals shifted 90° out of phase.

6. The surgical thread according to claim 1, characterized in that there is provided at least one repetition unit, which is the portion of thread between two left and right ends, with a middle point Pm at which the thorns invert their own inclination giving rise to two opposite sequences of thorns directed to opposed directions.

7. The surgical thread according to claim 6, characterized in that the length of the repetition unit is between 2 cm and 60 cm.

8. The surgical thread according to claim 6, characterized in that the length of the repetition unit is constant all over the length of the thread.

9. The surgical thread according to claim 6, characterized in that the length of the repetition unit is not constant all over the length of the thread so as to increase or decrease the density of the thorns in some portions of the thread and to provide a differentiated gathering according to the type of soft tissue crossed.

10. The surgical thread according to claim 6, characterized in that each repetition unit is separated from the following unit by a short length of thread without thorns.

11. The surgical thread according to claim 10, characterized in that the section of the thread is constant apart from its shape.

12. The surgical thread according to claim 1, characterized in that its section is increasing and/or decreasing.

13. The surgical thread according to claim 1, characterized in that the pitch of the thorns on the axis of the thread is constant.

14. The surgical thread according to claim 13, characterized in that the pitch of the thorns is between 0.50 mm and 17.20 mm.

15. The surgical thread according to claim 1, characterized in that the pitch of the thorns is varying according of the Fibonacci's series referred to the order of magnitude of the thread's thickness.

16. The surgical thread according to claim 1, characterized in that it consists of a straight thread which is caused to take a permanent spiral shape with a well-defined inner diameter giving it elastic characteristics, the thorns of said thread having a straight or spiral course.

17. The surgical thread according to claim 1, characterized in that a spiral thread with a lower diameter is wound about the thread provided with thorns, the inner gauge of the spiral being at least the same as the diameter of the needle by which said thread is inserted into the tissues.

18. The surgical thread according to claim 1, characterized in that the length of the thorns or projections is between 0.3 and 5.5 mm.

19. The surgical thread according to claim 1, characterized in that the cutting angle of the thorns is between 10° and 70°.

20. The surgical thread according to claim 1, characterized in that the thorns have the shape of a fishbone.

21. The surgical thread according to claim 1, characterized in that the thorns have the shape of an ear of wheat.

22. The surgical thread according to claim 1, characterized in that the thorn has the shape of a microturn.

23. The surgical thread according to claim 1, characterized in that the thorns have a spiral course.

24. The surgical thread according to claim 1, characterized in that it is made of reabsorbable material.

25. The surgical thread according to claim 1, characterized in that it is made by die-casting.

26. The surgical thread according to claim 1, characterized in that it is made by applying nanotechnology techniques.
